(19) United States
(12) Patent Application Publication
(10) Pub. No.: US 2024/0398257 A1
(43) Pub. Date: Dec. 5, 2024

(54) METHOD FOR ESTIMATING A BODY SURFACE AREA OF A SUBJECT AND MEDICAL IMAGING DEVICE

(71) Applicant: Siemens Healthineers AG, Forchheim (DE)

(72) Inventors: Ralf GUTJAHR, Nuernberg (DE); Andreas WIMMER, Forchheim (DE); Alexander KATZMANN, Fuerth (DE)

(73) Assignee: Siemens Healthineers AG, Forchheim (DE)

(21) Appl. No.: 18/647,171

(22) Filed: Apr. 26, 2024

(30) Foreign Application Priority Data
May 30, 2023 (EP) 23176127.1

(51) Int. Cl. A61B 5/107; A61B 5/00; A61B 6/00; G06T 17/20

(52) U.S. Cl. CPC A61B 5/1075; A61B 5/744; A61B 6/488; G06T 17/20; G06T 2210/41

(57) ABSTRACT

A method and corresponding system of an imaging device for estimating a body surface area of a subject, the method comprising sampling the subject with at least one sensor device to acquire sample data of the subject; transferring the sample data to a processing device; and using the processing device, applying at least one algorithm to the sample data in order to determine the body surface area of the subject based on the sample data.

### CROSS-REFERENCE TO RELATED APPLICATION(S)

The present application claims priority under 35 U.S.C. § 119 to European Patent Application No. 23176127.1, filed May 30, 2023, the entire contents of which is incorporated herein by reference.

### FIELD

One or more example embodiments of the present invention relates to a method for estimating a body surface area of a subject such as a human being or animal, a corresponding computer program, a body surface estimation system, and a corresponding medical imaging device.

Independent of the grammatical term usage, individuals with male, female or other gender identities are included within the term.

### RELATED ART

Determining an appropriate drug dose for a patient for treatment and/or examination, e.g. for the application of a (chemo-) therapeutic agent or contrast media that may be used for medical imaging, is an important factor in medical workflows and patient outcome. A relatively simple and therefore convenient approach is a patient weight-based drug scaling. This approach can be particularly convenient because, for the determination of the drug-scaling, only the patient weight is needed. For this, the weight may be acquired from a hospital system such as a hospital information system (HIS) or a radiology information system (RIS) or by using a personal scale. Therefore, patient weight-based drug scaling is common practice in many clinical workflows. However, the drawback of this approach is a limited reliability and accuracy. For example, in particular when being based on information acquired from a hospital system, the weight information may be outdated and the patient's weight may have changed, e.g. due to illness effects. Furthermore, patient weight does not scale directly with the blood volume of the patient, while the blood volume is a determinant factor for drug scaling. Namely, this drug scaling based on patient weight does not consider that the respective drug traverses through the vascular system, visceral organs, and muscles to a greater extent than it does through body fat. Accordingly, the variances in body size and shape across different patients may not be taken into account by applying this weight-scaling based method. Particularly for obese patients this reduced contribution to dispersion and dilution of a drug can lead to overdosing, potentially resulting in an increased risk of impaired effectiveness of a therapy or suboptimal quality of image-based diagnosis, e.g. due to artifacts created due to overcontrasting. An alternative to using the patient weight as basis for drug scaling, is to use the Body Mass Index (BMI) of the patient. However, similar effects can occur when a drug scaling is determined by using a patient's BMI, since the BMI may also be heavily be influence by the amount of body fat, and thus, the BMI is usually more a measure of body fat rather than a correlating measure of a patient's blood volume.

It has been found that linear drug scaling based on the patient's body surface area (BSA) can be more precise and reliable than drug scaling being based on patient weight or BMI, since the BSA tends to be about linear with respect to the blood volume. Furthermore, even beyond drug scaling, the body surface area (BSA) can be an important variable to be used for diagnosis and treatment planning in the medical context, for example in the case of skin burn.

### SUMMARY

However, manual calculation of the BSA can be time consuming and hinder a smooth workflow. Furthermore, objects that obstruct the view of the patient, such as clothes, respirators or tubes, may make it even more difficult to manually determine the BSA. For example, a systematic overestimate be a consequence. This may be especially critical in emergency situations, e.g. involving trauma. The BSA can be estimated using one of many different equations as proposed in early and recent literature. Most of these formulas rely on patient variables such as height, weight, and sometimes even sex and age. However, these formulas tend to be lacking additional variables indicative of physical or other characteristic variations, in particular concerning individual particularities of a patient, such as dwarfism, gigantism, anorexia, amputation. Furthermore, gathering the necessary variables for these formulas may still be a relatively laborious task.

One or more example embodiments provides a means to determine the body surface area in a more automatic and preferably more reliable way, preferably while being adaptable to individual patient properties.

This is met or exceeded by a method according to claim 1, a computer program according to claim 13, a body surface estimation system according to claim 14, and a medical imaging device according to claim 15.

### BRIEF DESCRIPTION OF THE DRAWINGS

The accompanying drawings illustrate various exemplary embodiments and methods of various aspects of the invention.

FIG. 1 shows a flow diagram of a method for estimating a body surface area of a subject according to an embodiment of the invention;

FIG. 2 shows a flow diagram of a method for estimating a body surface area of a subject according to another embodiment of the invention;

FIG. 3 shows a flow diagram of a method for estimating a body surface area of a subject according to another embodiment of the invention;

FIG. 4 shows a flow diagram of a method for estimating a body surface area of a subject according to another embodiment of the invention;

FIG. 5 shows a surface image reconstructed from three-dimensional sample data sampled with a 3D camera;

FIG. 6 shows an avatar model that is fitted to sample data with a method according to an embodiment of the invention;

FIG. 7 shows a zoom-in on the area marked in FIG. 6 by a dashed box;

FIG. 8 shows an avatar model according to an embodiment of the invention shown together with part of a reconstruction of sample data;

FIG. 9 shows the network architecture of a residual convolutional neural network according to an embodiment of the invention; and

FIG. 10 shows a medical imaging device comprising a body surface estimation system for estimating the body surface area of a subject according to an embodiment of the invention.

### DETAILED DESCRIPTION

According to one or more example embodiments of the present invention, a method for estimating a body surface area of a subject, in particular a patient, is provided. The subject may, for example, be a human being or an animal. The method comprises the following steps:
(a) sampling the subject with at least one sensor device to acquire sample data, in particular three-dimensional sample data, of the subject;
(b) transferring the sample data to a processing device;
(c) on the processing device, applying at least one algorithm to the sample data in order to determine the body surface area of the subject based on the sample data.

Hence, the inventive concept is to use sample data of the subject that is acquired by the sensor device, in order to determine the body surface area (BSA) of the subject. The sensor device may advantageously be a sensor device that is already present at a clinical side, since sensor devices are often included in medical examination rooms. The sensor may be a three-dimensional (3D) sensor or a two-dimensional (2D) sensor. A 3D sensor is e.g. a 3D camera, whereas a 2D sensor may be an ordinary (2D) optical camera. On the other hand, it may be relatively easy and inexpensive to add such sensor device to the clinical site. Advantageously, the sampling of the subject via the sensor device may be performed automatically or semi-automatically, e.g. by manually activating the sensor device and then automatically taking and transferring the sample data. Accordingly, the effort for medical staff may be kept low. Furthermore, the BSA can be obtained quickly and reliably during a regular clinical workflow. Potentially, besides the steps of a usual clinical workflow, the subject does not even have to perform any additional actions, such as stepping on a scale, taking a certain pose or undress. Optionally, an additional step may be added to the method of planning a treatment and/or determining a drug scaling, such as contrast agent scaling, based on the determined body surface area. Optionally, the determined body surface area may be transferred to another device, such as an imaging device, in particular medical imaging device, a contrast injector, a hospital information system and/or a radiology information system. Imaging devices may, for example, be a magnetic resonance tomography system, a computed tomography system and/or an ultrasound system.

The sample data may, for example, be transferred to the processing device via a data cable or via a wireless connection. The processing device may be or may comprise, for example, a computer, such as a personal computer, a cloud computer, a server. Additionally, and/or alternatively, the processing device may be the processing unit of an imaging system. Advantageously, it has been found that the sample data can be enough information for the algorithm that is applied in order to determine a reliable and accurate estimation of the BSA. There are different alternatives of algorithms that may be applied. Corresponding embodiments are given below. Optionally, more than one algorithm may be applied. At least two algorithms may be applied consecutively or simultaneously. For example, it is conceivable that two algorithms are applied simultaneously and a further algorithm is applied afterwards or before. At least two different algorithms may determine the BSA essentially independently of each other. A further algorithm may compare the outcome of the two different algorithms or determine a BSA based on the outcome of the two different algorithms. Advantageously, an even greater reliability may be achieved by such a combination of independent algorithms.

The sample data may in particular comprise optical, acoustical, electromagnetic, topographic and/or tomographic data of the subject. Accordingly, the sensor device may, for example, be an optic sensor device, an acoustic sensor device, an electromagnetic sensor device, or a topographic sensor device. The sensor device is in particular configured to sample the subject's surface, typically a part of the subject's surface, in particular one side of the subject, namely the subject facing the sensor device. Typically, the subject will lie on a patient table, and the sensor device in configured to acquire sample data from above, capturing the upper side or part of the upper side of the subject. The sensor device may automatically take the sample data of the subject. For example, the sensor device may be configured to automatically take the sample data at a defined step of a clinical workflow. A defined step may for example comprise the patient lying on a patient table, such as a patient table of a medical imaging system, or the patient entering an examination room, e.g. through a door. Optionally, plurality of sensor devices may be used to independently acquire a plurality of sets of three-dimensional sample data. Advantageously, redundant information may be used to minimize an overall error due to individual accuracies of the sensor devices. In particular, complementary sample data may be fused together. Complementary sample data may, for example, be sample data measured from different angles with respect to the subject.

According to an embodiment, the at last one sensor device comprises an optical sensor, in particular a 3D camera, and/or a radar sensor and/or an acoustic sensor. The optical sensor may, for example, be an optical camera, in particular optical 3D camera, or a laser sampling device such as based on LiDAR (Light Detection and Ranging). The acoustic sensor may, for example, be based on ultrasound. A 3D camera may advantageously allow to directly acquire the three-dimensional sample data, in particular with a stationary sensor device. A sampling based on LiDAR may be performed by sending light pulses and detecting signals that are reflected by the subject. The runtime of the light may be used to calculate the distance to each part of the subject the light is reflected from. The light pulses may, for example be laser pulses or infrared light pulses. Preferably, based on the calculated distance, the shape of the subject is determined. The optical sensor may comprise a structured light scanner and be adapted to cast structured light on the subject. The structured light may in particular be a structured pattern, for example a grid, and the sensor device may be adapted to project the structured pattern on the subject. The structured light scanner may be configured to measure a distortion of the structured pattern that is projected on the subject. A position of different surface points of the subject may be calculated from the measured structured pattern on the subject, in particular in order to determine a shape of the subject.

According to an embodiment, the three-dimensional sample data is acquired by moving the sensor device or the subject, by triangulation, or by applying a time-of-flight measurement of the subject by the sensor device. In particular, the sensor device may comprise at least one two-dimensional sensor. The at least one two-dimensional sensor may be adapted to acquire three-dimensional sample data by moving into different positions relative to the subject and acquire position dependent sample data of the subject. The position-dependent sample data may be used to determine three-dimensional sample data. For performing triangulation, at least two sensor devices, in particular two-dimensional sensor devices, may be provided. The time-of-flight measurement may, for example be based on radar (radio detection and ranging) or LiDAR. The time-of-flight measurement may be based on sending radiation, in particular infrared light, and calculate the phase difference of reflected radiation.

According to an embodiment, a medical imaging device, in particular an X-ray based medical imaging device, is used as a or the sensor device that provides medical image data as sample data or as part of the sample data, in particular such that a pre-scan taken by the medical imaging device is used as medical image data. The pre-scan may for example be a topogram that is acquired before a main examination scan. The pre-scan may be a preliminary non-contrast scan. The X-ray-based imaging device may in particular be a computed tomography scanner. The sample data may, for example, be acquired from a topographic X-ray scan or a planar X-ray scan, for example in lateral and/or anterior-posterior direction. Advantageously, data that is to be measured anyway during a medical workflow may be used for the sample data. The acquisition of sample data may such be integrated into a medical workflow essentially seamlessly. Optionally, the sample data from the medical imaging device may be used to support sample data acquired with another sensor device. Hence, the sample data from the medical imaging device and another sensor device may be used as redundant information in order to minimize an overall error the data. The sample data from the medical imaging device may be used to adjust sample data acquired with another sensor device.

According to an embodiment, determining the body surface area of the subject comprises:
creating a virtual avatar model of the subject's surface based on the sample data and a statistical shape model, such that the shape and pose of the avatar model is a fit to the sample data, wherein the fit adheres to boundary conditions defined by the statistical shape model,
   wherein the statistical shape model comprises a database of a range of standard subject shapes and poses that define a range of shapes and poses the avatar model is bound to adhere to;
calculating the surface area of the avatar model and using the calculated surface area of the avatar model as estimate for the body surface area.

The avatar model is in particular a model that describes the surface of the subject. The applied algorithm may in particular be configured to determine the avatar model such that the avatar model assumes both the pose and the body proportions of the observed subjects. In particular, the avatar model is determined such that it adheres to the statistical shape model. The statistical shape model may preferably be based on a database of body variations of real subjects, in particular real human beings or animals. For example, the avatar model may be determined as described by Singh, Vivek & Ma, Kai & Tamersoy, Birgi & Chang, Yao-Jen & Wimmer, Andreas & O'Donnell, Thomas and Chen, Terrence in “DARWIN: Deformable Patient Avatar Representation With Deep Image Network”, 497-504, 10.1007/978-3-319-66185-8_56; 2017. The statistical shape model may preferably comprise shapes and positions that comprises a plurality of characteristic subject variations. Characteristic subject variations may comprise individual properties of different subjects, such as the subject being large or small. Individual properties may preferably comprise extreme properties such as including being extraordinarily small or large, being particularly thin or overweight and/or missing limbs. It has been found that the application of such an avatar model as intermediate step in order to calculate the surface area can lead to accurate and reliable results. Furthermore, the applied algorithm may only need a relatively low amount of training data, compared to a trained algorithm that is trained to directly determine a surface area, to achieve useful results of surface area determination. Furthermore, it has been found that the surface area can thus be determined quickly, in particular such that it can be integrated within a clinical workflow, usually without causing any detrimental delays of the workflow.

According to an embodiment, creating the virtual avatar model comprises allocating landmarks to at least some characteristic parts of the subject in the sample data, in particular by applying a first trained neural network, wherein the avatar model is created by mapping the avatar model to the landmarks, in particular by applying a second trained neural network. Characteristic parts may comprise defined parts of the head, such as the top and/or bottom of the head, shoulders, elbows, wrists, torso, such as the center of the torso, groin, knees and ankles. For example, 10 to 20 landmarks may be used. The first and/or second trained neural network may preferably be a convolutional neural network. The first/second neural network may in particular be configured and trained as described by Singh, Vivek & Ma, Kai & Tamersoy, Birgi & Chang, Yao-Jen & Wimmer, Andreas & O'Donnell, Thomas and Chen, Terrence in “DARWIN: Deformable Patient Avatar Representation With Deep Image Network”, 497-504, 10.1007/978-3-319-66185-8_56; 2017.

According to an embodiment, the first trained neural network is trained to allocate landmarks despite the subject being at least partially covered by covering elements, in particular by clothes and/or other objects; wherein the first trained neural network is in particular trained such that the applied training data comprise at least some input sample data in which the subject is at least partially covered by the covering elements. Covering elements may in particular comprise clothes and/or blankets. Covering elements may also comprise objects such as oxygen tanks or parts of a medical imaging device. Hence, advantageously, this embodiment may allow to realistically estimate the surface area of the subject without the need for the subject to undress. An existing medical workflow may advantageously not have to be adapted, such as by removing a blanket or by undressing. This may save time during a workflow. Furthermore, not needing to undress may be particularly advantageous for persons or patients that are sick and/or weakened and may thus have trouble to undress. The first trained neural network may be trained with datasets comprising different subjects with different coverings. The training data may comprise sample data of subjects in different positions. The training data may preferably comprise sample data with and without coverings, in particular such that the training data comprise sample data, wherein a same subject in a same pose is comprised with and without cover.

According to an embodiment, after mapping the avatar model to the landmarks and before calculating the surface area, a model-fitting algorithm is applied such that the avatar model is adapted to better fit to the sample data within the boundaries defined by the statistical shape model, wherein the statistical shape model in particular comprises degrees of freedom allowing to modulate joints and a current orientation of joints of the subject. Thus, preferably, three algorithms or one algorithm with three parts, may be applied. The first algorithm of first part of the algorithm may define landmarks, the second algorithm or second part of the algorithm may perform a mapping of the avatar model to the landmarks, and the third algorithm or third part of the algorithm, i.e. the model-fitting algorithm, may adjust the avatar model. Optionally, the model-fitting algorithm may be configured to adhere to degrees of freedom within which joints of the avatar model may be modulated. Joints of the avatar model may, for example, comprise one, multiple or all of shoulders, elbows, wrists, knees and ankles of the subject.

According to an embodiment, the avatar model is or is represented by a three-dimensional geometric mesh of the body surface, in particular a mesh of triangles. The mesh can be well-suited to be used in order to calculate the surface. For example, the coordinates of individual elements of the mesh, in particular corner points, may be used to calculate the area of the individual elements. The area of the individual elements may be added up in order to calculate the full surface area of the subject. The elements of the mesh may in particular be the cells of the mesh. It has been found that a mesh of triangles can be particularly advantageous in order to calculate the surface area reliably and accurately. For example, the mesh may comprise a number N of triangles, Ti, each triangle being defined by three corner points, vi,1, vi,2, vi,3. The corner points may be vectors defining the position of the corner points. The area of the elements of the mesh may be calculated by applying a vector product of coordinates defining the elements. The BSA may be calculated based on the areas of the elements, in particular by adding up the areas of the elements. For example, the individual area of a triangle Ti may be calculated via the formula

Ai=0.5(vi,2−vi,1)×(vi,3−vi,1),

x being the vector product of the vectors. Accordingly, the surface area BSA of the avatar model may be determined via the calculation

$$BSA = {\sum }_{i = 1}^{N} {A}_{i}$$

According to an embodiment, applying at least one algorithm comprises applying a convolutional neural network (CNN) that is trained to derive an estimate for the body surface area based on the sample data. This embodiment may in particular be useful, when a large amount of training data is available. For example, when a training database with an amount of data from subjects in the order of magnitude of hundred thousand is available, this embodiment can provide particularly reliable and robust results. Accordingly, for this embodiment, an avatar model does not have to be determined, but the surface area may be determined directly from the sample data. The CNN may be a residual convolutional neural network. For example, the CNN may comprise a series of residual blocks, each residual block consisting of a series of convolutional blocks. Each convolutional block may consist of a sequence of a convolution operator followed by an activation function, followed by a normalization function. Normalization layers may ensure an efficient training. Residual connections between convolutional blocks may ensure a sufficient gradient flow throughout the network. Each residual block may be followed by an average pooling operator. The CNN may preferably comprise a fully connected layer performing a body surface regression and producing a single output in the form of a body surface area. The output surface area may be a z-normalized BSA estimate.

The CNN may be trained via supervised training. The CNN may be trained by using paired data tuples, e.g. <x, y>. The data tuples may each comprise an input image, e.g. x, and a target variable, e.g. y. The input image may be derived from a sensor device, such as an optical or acoustical sensor. The target variable is in particular the surface area (BSA). Hence the target variable may be the ground truth of the training process. The BSA used for training may be measured or estimated by established formula, e.g. based on patient weight and height. Alternatively, the BSA of the target variables may be estimated based on a subject topogram. The subject topogram may provide an exact measurement of the patient surface. Alternatively, the target variable may be derived based on any of the embodiments employing the avatar model. Hence, the embodiment of the avatar model, requiring less training data, may be used to build up a training database for this embodiment. Some or all of the alternatives for acquiring target variables as described here may be combined, e.g. in order to acquire a larger amount of training data via different means. Advantageously, it has been found that, during training, the CNN may derive relevant criteria, such as defined by the statistical shape model according to other embodiments, automatically. Given a large enough amount of training data, the CNN may be able to outperform the quality of the training data, in particular by generalizing BSA factors other than patient weight and height, such as less common patient geometries or missing limbs.

According to an embodiment, prior to applying the convolutional neural network, an orthographic projection of the sample data is generated and the convolutional neural network is applied to the orthographic projection. It has been found that an orthographic projection may lead to more reliable results, since the input into the CNN may thus be independent on the specific positioning of the sensor device, in particular with regard to a distance between the sensor device and the subject. Hence, by applying the orthographic projection, the input data into the CNN may be standardized and the CNN thus may have to deal with a smaller range of different data. Preferably, the orthographic projection is always performed from essentially the same view angle with respect to the subject.

According to an embodiment, the body surface area is calculated based on the estimate of both the convolutional neural network and the estimate derived via the avatar model, in particular by determining an average or a weighted average of both estimates. Preferably, the two estimation methods may each be applied independently on the same sample data and the results may be combined afterwards. This combination may lead to more accurate and reliable results. A weighted average may in particular be applied when it is found that one of the two estimates tends to be more accurate. In this case the weighting may be such that more weight is put on the more accurate alternative. The weighting may be determined by further factors. For example, the weighting may depend on subject properties. This may, for example, be advantageous when one of the independent estimates is more reliable for some particular properties but less reliable for other particular properties.

According to an embodiment, the estimate of the body surface area derived from the convolutional neural network is compared to the estimate of the body surface area derived via the avatar model, wherein, in case that a difference in the estimated body surface area is found to be greater than a pre-defined threshold, a warning is sent to a user. Advantageously, two independently derived BSA may be used to make a reliability check. Hence, when the different of the two estimated BSA is greater than a threshold, it may be assumed that at least one estimate is faulty. It may then be advantageous to repeat the whole method, in particular with new sample data or to discard one of the estimates.

According to one or more example embodiments of the present invention, a computer program comprising instructions which, when the program is executed on a system comprising a processing device and at least one sensor, cause the system to carry out the steps of the method as described herein. All features and advantages of the method may be adapted to the computer program and vice versa.

According to one or more example embodiments of the present invention, a body surface estimation system is provided. The body surface estimation system comprises at least one sensor device, in particular a sensor device as described herein. The body surface estimation system further comprises a processing device configured to receive sensor signals from the at least one sensor device. Optionally, the body surface estimation system may comprise an output device configured to output derived information from the processing device. The processing device is configured to apply at least one algorithm in order to determine the body surface area of the subject based on the sample data. In particular, the body surface estimation system may be configured to carry out the method steps of the method for estimating a body surface area of a subject as described herein. All features and advantages of the method and of the body surface estimation system may be adapted to the computer program and vice versa.

According to one or more example embodiments of the present invention, a medical imaging device comprising a body surface estimation system as described herein is provided. All features and advantages of the method, of the body surface estimation system, and of the computer program may be adapted to the medical imaging device and vice versa.

The embodiments described herein may be combined with each other unless indicated otherwise.

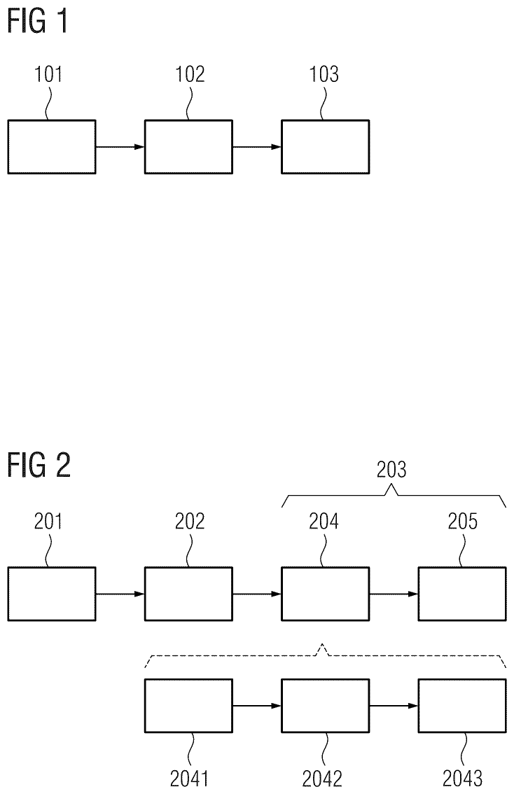

FIG. 1 shows a flow diagram of a method for estimating a body surface area of a subject 5 according to an embodiment of the invention. In a first step 101, the subject 5 is sampled with at least one sensor device 2 to acquire three-dimensional sample data 20 of the subject 5. In this embodiment and in any other embodiment, the sensor device 2 may comprise an optical sensor such as a 3D camera and/or an acoustic sensor. Optionally, the sample data 20 may be acquired by triangulation, by moving the sensor device 2, or by applying a time-of-flight measurement of the subject 5 by the sensor device 2. Additionally or alternatively, the sample data 20 or some of the sample data 20 may be provided by a medical imaging device 1. Hence, the medical imaging device 1 may be used as sensor device 2. For example, a pre-scan performed by the medical imaging 1 device may be used as sample data 20. In a further step 102 the sample data 20 is transferred to a processing device 3. The processing device 3 may, for example, be part of a control unit of the medical imaging device 1. In a further step 103, on the processing device 3, at least one algorithm is applied in order to determine the body surface area of the subject 5 based on the sample data 20.

FIG. 2 shows a flow diagram of a method for estimating a body surface area of a subject 5 according to another embodiment of the invention. In a first step 201, the subject 5 is sampled with at least one sensor device 2 to acquire three-dimensional sample data 20 of the subject 5. In a further step 202 the sample data 20 is transferred to a processing device 3. A further step 203 of applying at least one algorithm in order to determine the body surface area of the subject 5 based on the sample data 20 comprises two sub-steps 204, 205. Namely, a first sub-step 204 comprises creating a virtual avatar model of the subject's surface based on the sample data 20 and a statistical shape model. The statistical shape model comprises a database of a range of standard subject 5 shapes and poses that define a range of shapes and poses the avatar model is bound to adhere to. Accordingly, the avatar model is created such that the shape and pose of the avatar model is a fit to the sample data 20 that adheres to boundary conditions defined by the statistical shape model. Preferably, the avatar model may be a three-dimensional geometric mesh 11 of the body surface. Optionally, the first sub-step 204 may comprise consecutive steps 2041-2043. A first consecutive step 2041 of the consecutive steps 2041-2043 comprises allocating landmarks to at least some characteristic parts of the subject 5 in the sample data 20. A further consecutive step 2042 comprises mapping the avatar model to the landmarks in order to create the avatar model. Both, step 2041 and step 2042 may preferably each be performed by applying a neural network, in particular a convolutional neural network. Preferably, the trained neural network used for the step 2041 of allocating landmarks is trained to allocate landmarks despite the subject 5 being at least partially covered by covering elements such as clothes, blankets and/or other objects. In a further consecutive step 2043, performed after step 2042, a model-fitting algorithm is applied such that the avatar model is adapted to better fit to the sample data 20 within the boundaries defined by the statistical shape model. For this purpose, the statistical shape model may comprise degrees of freedom that allow to modulate joints and a current orientation of joints of the subject 5. In a further step 205, the surface area of the avatar model, in particular the adapted avatar model, is calculated and the calculated surface area of the avatar model is used as an estimate for the body surface area.

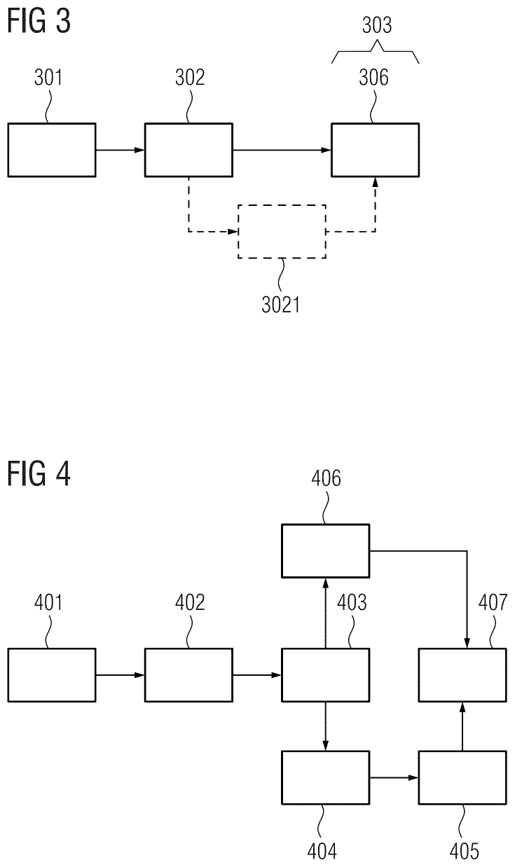

FIG. 3 shows a flow diagram of a method for estimating a body surface area of a subject 5 according to an embodiment of the invention. In a first step 301, the subject 5 is sampled with at least one sensor device 2 to acquire three-dimensional sample data 20 of the subject 5. In a further step 302, the sample data 20 is transferred to a processing device 3. On the processing device 3, at least one algorithm is applied 303 in order to determine the body surface area of the subject 5 based on the sample data 20. The step of applying an algorithm comprises or consists of a step 306 of applying a convolutional neural network that is trained to derive an estimate for the body surface area based on the sample data 20. In an optional step 3021, prior to the step 306 of applying the convolutional neural network, an orthographic projection of the sample data 20 may be generated and during step 306, the convolutional neural network may be applied to the orthographic projection.

FIG. 4 shows a flow diagram of a method for estimating a body surface area of a subject 5 according to an embodiment of the invention. In a first step 401, the subject 5 is sampled with at least one sensor device 2 to acquire three-dimensional sample data 20 of the subject 5. In a further step 402 the sample data 20 is transferred to a processing device 3. Optionally, in this step 402, the sample data 20 may be transferred to two different processing devices 3, in particular when the following steps 404 and 406 are performed on different processing devices 3. On the processing device 3 or on the two different processing devices 3, in a further step 403 at least two algorithms are applied to independently determine the body surface area of the subject 5 based on the sample data 20. In one step 406, a convolutional neural network is applied that is trained to derive an estimate for the body surface area based on the sample data 20. This step may in particular be performed as described with respect to FIG. 3, e.g. optionally including an orthographic projection of the sample data 20. In another step 404, an avatar model of the subject's surface based on the sample data 20 and a statistical shape model is created. The statistical shape model comprises a database of a range of standard subject shapes and poses that define a range of shapes and poses the avatar model is bound to adhere to. Accordingly, the avatar model is created such that the shape and pose of the avatar model is a fit to the sample data 20 that adheres to boundary conditions defined by the statistical shape model. In a further step 405, the surface area of the avatar model, in particular the adapted avatar model, is calculated and the calculated surface area of the avatar model is used as an estimate for the body surface area. These steps 404 and 405 may in particular be performed as described with respect to FIG. 2. In a further step 407, the body surface area is calculated based on the estimate of the route of step 404 and step 405 as well as the estimate of step 406. In particular, this step 407 may comprise determining an average or a weighted average of both estimates.

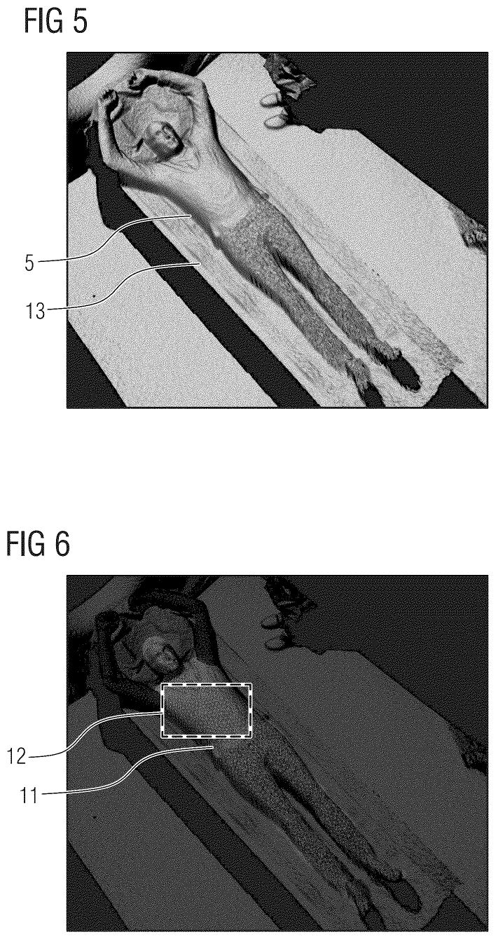

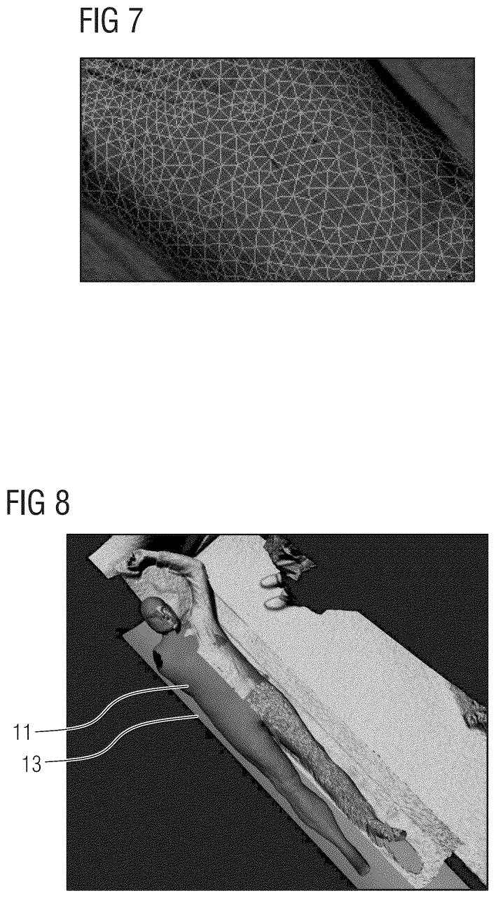

FIG. 5 shows a surface image reconstructed from three-dimensional sample data 20 sampled with a 3D camera. On this image, a subject 5 can be seen lying on a patient table 13. In FIG. 6, an avatar model is fitted to the sample data 20 with a method according to an embodiment of the invention. The avatar model comprises a three-dimensional geometric mesh 11 of the body surface. FIG. 7 shows a zoom-in on the area marked in FIG. 6 by a dashed box 12. As can be seen, the geometric mesh 11 is a mesh of triangles. The area of the individual triangles may be calculated by via a vector product of the coordinates of the corners of the triangles. The full body surface area may then be calculated by adding the calculated areas of the individual triangles. FIG. 8 shows the avatar model being shown together with part of the reconstruction of the sample data 20. As can be seen, the avatar model is fitted to the subject's body such that clothes of the subject 5 are actually placed above the avatar model. This shows that the avatar model as determined by an embodiment of the invention is not a fit to the outer clothes covering the subject 5 but to the surface of the actual subject 5. Hence, it can be seen that the method according to one or more example embodiments of the present invention is capable of accurately calculating the surface area of the subject 5, even though the subject 5 is partially covered with clothes. Furthermore, the avatar model follows the surface of the subject 5 above a patient table 13, thus being able to subtract the patient table 13 from the determination of the body surface area.

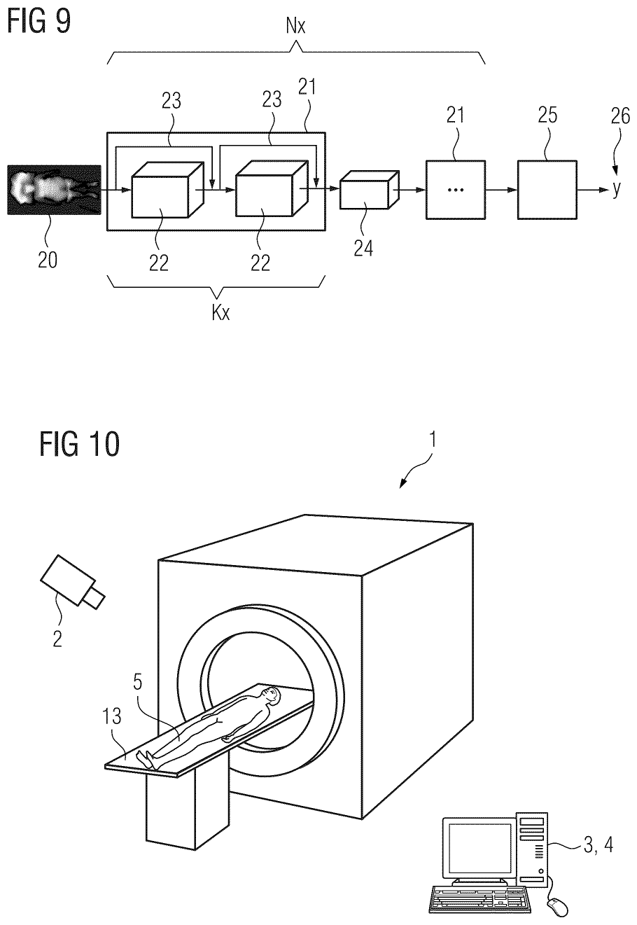

FIG. 9 shows the network architecture of a residual convolutional neural network according to an embodiment of the invention. This convolutional neural network is configured to determine the body surface area of a subject 5 without the need of calculating a virtual avatar model based on three-dimensional sample data 20 of the subject 5. The network architecture consists of a series of N residual blocks 21 of K subsequent convolutional blocks 22 with residual connections 23. The residual connections 23 between convolutional blocks 22 may ensure a sufficient gradient flow throughout the network. Each convolutional block may consist of a sequence of a convolution operator followed by an activation function, followed by a normalization function. After each residual block, an average pooling operator 23 is applied (here shown only after the first residual block 23). The average pooling operator 23 reduces the image by half in each dimension (indicated by the smaller residual block 21 after the average pooling operator 23. Finally, a fully-connected layer 25 is appended, resulting in a BSA estimate 26 for the sample data 20 of the subject 5.

FIG. 10 shows a medical imaging device 1 comprising a body surface estimation system for estimating the body surface area of a subject 5 according to an embodiment of the invention. The system comprises a sensor device 2 such as a 3D camera. The sensor device 2 is in particular configured to detect sample data 20 from the subject 5 lying on a patient table 13. The system further comprises a processing device 3 that is configured to receive sensor signals, in particular sensor signals transferring the sample data 20, from the sensor device 2. The processing device 3 is configured to apply at least one algorithm in order to determine/estimate the body surface area of the subject 5 based on the sample data 20. The estimation of the body surface area may, for example be carried out as described in any of the embodiments of FIGS. 1 to 4. The system may also comprise an output device 4 that is configured to output information derived by the processing device 3.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for estimating a body surface area of a subject, the method comprising:
sampling the subject with at least one sensor device to acquire sample data of the subject;
transferring the sample data to a processing device; and
using the processing device, applying at least one algorithm to the sample data in order to determine the body surface area of the subject based on the sample data.

2. The method of claim 1, wherein the determining the body surface area of the subject comprises:
creating a virtual avatar model of a surface of the subject based on the sample data and a statistical shape model, such that a shape of the virtual avatar model and a pose of the virtual avatar model is a fit to the sample data, the fit adhering to boundary conditions defined by the statistical shape model, the statistical shape model including a database of a range of standard subject shapes and poses that define a range of shapes and poses the virtual avatar model is bound to adhere to; and
calculating a surface area of the virtual avatar model and using the calculated surface area of the virtual avatar model as an estimate of the body surface area.

3. The method of claim 2, wherein the creating the virtual avatar model comprises:
allocating landmarks to at least some characteristic parts of the subject in the sample data using a first trained neural network, the creating creates the virtual avatar model by mapping the virtual avatar model to the landmarks using a second trained neural network.

4. The method of claim 3, wherein
the first trained neural network is trained to allocate the landmarks despite the subject being at least partially covered by covering elements, and
the first trained neural network is in particular trained such that the applied training data comprise at least some input sample data in which the subject is at least partially covered by the covering elements.

5. The method of claim 3, wherein
after mapping the virtual avatar model to the landmarks and before calculating the surface area of the virtual avatar model, a model-fitting algorithm is applied such that the virtual avatar model is adapted to fit to the sample data within the boundaries defined by the statistical shape model.

6. The method of claim 2, wherein the virtual avatar model is a three-dimensional geometric mesh of the body surface.

7. The method of claim 2, wherein the applying the at least one algorithm applies a convolutional neural network trained to derive an estimate for the body surface area based on the sample data.

8. The method of claim 7, wherein, prior to the applying the convolutional neural network, an orthographic projection of the sample data is generated and the convolutional neural network is applied to the orthographic projection.

9. The method of claim 7, wherein the calculating the body surface area is based on the estimate of both the convolutional neural network and the estimate derived via the virtual avatar model, in particular by determining an average or a weighted average of both estimates.

10. The method of claim 1, wherein the at last one sensor device comprises at least one of an optical sensor or an acoustic sensor.

11. The method of claim 1, wherein three-dimensional sample data is acquired by triangulation, by moving the sensor device or the subject, or by applying a time-of-flight measurement of the subject by the sensor device.

12. The method of claim 1, wherein a medical imaging device, in particular an X-ray based medical imaging device, is used as sensor device that provides medical image data as sample data or as part of the sample data, in particular such that a pre-scan taken by the medical imaging device is used as medical image data.

13. A computer program comprising instructions which, when executed on a system comprising a processing device and at least one sensor, cause the system to perform the method of claim 1.

14. A body surface estimation system comprising:
at least one sensor device;
a processing device configured to receive sensor signals from the at least one sensor device; and
an output device configured to output derived information from the processing device, wherein the processing device is configured to cause the system to perform the method of claim 1.

15. A medical imaging device comprising:
the body surface estimation system of claim 14.

16. The method of claim 1, wherein the sample data is three-dimensional sample data of the subject.

17. The method of claim 5, wherein the statistical shape model comprises degrees of freedom allowing to modulate joints and a current orientation of joints of the subject.

18. The method of claim 6, wherein the virtual avatar model is a mesh of triangles.

19. The method of claim 9, wherein the calculating includes determining an average of the estimate of both the convolutional neural network and the estimate derived via the virtual avatar model or a weighted average of the estimate of both the convolutional neural network and the estimate derived via the virtual avatar model.

20. The method of claim 12, wherein the medical imaging device is an X-ray based medical imaging device and a pre-scan taken by the medical imaging device is used as medical image data.
